(19) United States
(12) Patent Application Publication
(10) Pub. No.: US 2019/0038461 A1
(43) Pub. Date: Feb. 7, 2019

(54) APPARATUS FOR CATARACT SURGERY

(71) Applicant: Chandrashekar BALACHANDRAN, Killara (AU)

(72) Inventor: Chandrashekar BALACHANDRAN, Killara (AU)

(21) Appl. No.: 16/050,166

(22) Filed: Jul. 31, 2018

(30) Foreign Application Priority Data
Aug. 1, 2017 (AU) 2017903031

(51) Int. Cl. A61F 9/007

(52) U.S. Cl. CPC A61F 9/00754; A61F 9/008; A61F 9/00745

(57) ABSTRACT

A method for removing a lens from an eye comprising the steps of making a hole in the outer eye to provide access to a lens capsule within the eye, cutting at least one discrete slit in the lens capsule to provide access to the lens, the slit being suitable to receive a tool to extract the lens and removing the lens through the slit.

This application claims benefit of Serial No. 2017903031, filed 1 Aug. 2017 in Australia and which application is incorporated herein by reference. To the extent appropriate, a claim of priority is made to the above disclosed application.

The present invention relates to a method and apparatus for performing cataract surgery and, in particular, a method and apparatus for removing a lens from an eye and inserting an artificial lens into the eye.

### BACKGROUND OF THE INVENTION

Cataract surgery has for the last 50 years been performed by phacoemulsification. This and the previous technique of extracapsular cataract surgery were characterised by the opening of the crystalline lens capsule to extract the lens material.

The lens consists of a central harder nucleus and a surrounding epi-nucleus and cortex.

During extracapsular cataract extraction surgery (ECCE surgery) a large opening is made in the anterior lens capsule know as a can-opener capsulotomy. Through this large opening and with the aid of a large corneal incision, the lens is expressed. The residual cortex is removed and a lens is placed in the remaining capsular bag. The technique is associated with limited control.

During this process, there is a risk of the crystalline material causing trauma to the irreplaceable corneal endothelium, which is critical for maintaining corneal transparency. The corneal endothelial damage manifests as corneal edema and can in a compromised cornea cause permanent swelling and vision loss.

The introduction of phacoemulsification reduced the trauma to the corneal endothelium. Phacoemulsfication involves applying ultrasound to emulsify the crystalline lens in the capsular bag. However, the acoustic wave generated can pass freely in all directions. In particular, the corneal endothelium which is usually about 3 mm or less from the phacoemulsification handpiece tip can be damaged. The amount of corneal endothelial damage is proportional to the amount of ultrasound energy used and the distance of the ultrasound tip from the corneal endothelium. The more the dense the cataract i.e. the nucleus, the more ultrasound energy needed. The ultrasound energy used is quantified as cumulative dissipated energy (CDE); a product of the power of the ultrasound and the duration of application inside the eye.

Attempts have been made to minimise the amount of corneal endothelial exposure to the ultrasound. The first strategy involves the use of ophthalmic viscoelastic device (OVD). A dispersive visocoleastic OVD in the anterior chamber is used to coat the corneal endothelium and a cohesive OVD is injected deeper in the anterior chamber. This is called a soft-shell technique. This strategy dampens the acoustic wave that hits the corneal endothelium. This technique as part of phacoemulsification has limitations. The ultrasound used during phacoemulsification generates heat. A constant flow of fluid is used to absorb the heat and prevent the temperature from rising inside the eye. Unfortunately, the circulating fluid washes out the transparent OVD increasing the exposure of the corneal endothelium to acoustic waves. The surgeon is unable to detect this OVD loss during surgery, as OVD is transparent and the replacement OVD is expensive.

The second strategy has been to fragment the crystalline lens into small pieces using various manual chopping techniques or recently using lasers. This allows the small lens fragments to be removed using the same phacoemulsification handpiece with little to no ultrasound energy. This has significantly reduced the CDE from 7 to 3 in mild to moderate cases. However, dense lenses remain a challenge. The goal of zero CDE loss remain elusive.

Attempts have been made to place a mechanical barrier in the form of contact lenses intraocularly in rabbit eyes. This idea though novel will likely not work in humans due to the turbulence induced by the phacoemulsification which would make the contact lens ricochet against the corneal endothelium causing significant damage to the corneal endothelium. An attempt has been made to use the complete capsulotomy made with the laser as a barrier. OVD is injected deep to the free-floating capsulotomy and the capsulotomy is floated to the corneal endothelium. The 2 are separated by a thin uncontrolled layer of OVD, again risking direct trauma to the endothelium from the capsular disc if the intervening transparent OVD is inadvertently absorbed. Furthermore, since the capsule is not tethered to any anatomical structures it tends to slide around and its protection of the corneal endothelium is not assured. The free-floating capsulotomy is often eccentric or can be aspirated inadvertently into the phacoemulsification handpiece thereby not protecting the central corneal endothelium.

Attempts have also been made to decrease the size of the capsulotomy by making smaller circles. Unfortunately these small capsulotomy/capsulorhexis limit the movement of the phacoemulsification handpiece and can tear the anterior capsule by stretching it potentially resulting in the lens dropping to the retina. Furthermore, more than 1 small circular aperture will be needed for the handpiece and a second manipulating or irrigating device. To connect 2 small circles in a lens capsule to form a larger circle or to independently use 2 small circles for intraocular lens insertion is a difficult challenge.

### SUMMARY OF INVENTION

In a first aspect the invention provides a method for removing a lens from an eye comprising the steps of:
making an incision in the outer eye to provide access to a lens capsule within the eye;
cutting at least one discrete slit in the lens capsule to provide access to the lens, the slit being suitable to receive a tool to extract the lens; and
removing the lens through the slit.

Embodiments of the invention provide the advantage that a capsulotomy including a large opening in the anterior lens capsule is not required. The maintenance of the integrity of the large portion of the anterior lens capsule assists with blocking of the acoustic waves from the phaco emulsification hand piece to limit the exposure of the corneal endothelium to the ultrasound.

Preferably, the at least one slit is positioned at a suitable distance away from the centre of the eye.

Maintenance of the integrity of the anterior lens capsule across the front of the eye maintains the ability of the lens zonules to be pulled by the ciliary body to control the thickness of the lens in order to maintain control of focal length i.e. accommodation.

In embodiments the at least one slit is positioned at a suitable distance from the centre of the eye.

In embodiments the at least one slit is positioned at a variable distance from the centre of the eye.

Preferably the at least one slit comprises at least two discrete slits. This allows multiple surgical instruments to be inserted into the lens capsule simultaneously from different locations to assist the surgical procedure.

Preferably the at least one slit is arced. In further embodiments, the slit can be of variable shapes such as an inverted T or a mouth shaped incision. This or any other shape would allow the instruments to manipulate the lens without tearing the capsule

In a second aspect the invention provides a method for removing a lens from an eye comprising the steps of:
making an opening the outer eye to provide access to a lens capsule within the eye;
cutting two or more discrete slits in the eye capsule, the slits being positioned away from the centre of the limbus of the eye, wherein at least one of the slits is suitable to receive a tool for extracting the lens; and
removing the lens through at least one of the slits.

The opening may also be used for inserting or injecting an intra-ocular lens.

In a third aspect the invention provides a laser configured for use in cataract surgery the laser being configured to cut at least one discrete slit in the lens capsule of an eye to provide access to the lens, the slit being suitable to receive a tool to extract the lens.

Preferably the at least one slit is positioned away from the centre of the limbus of the eye.

In embodiments the at least one slit is positioned at a fixed distance from the centre of the limbus of the eye.

In embodiments the at least one slit is positioned at variable distances from the centre of the limbus of the eye.

Preferably the at least one slit comprises at least two discrete slits.

Preferably the at least one slit is arched.

Preferably the laser is configured to emulsify the lens.

### BRIEF DESCRIPTION OF THE DRAWINGS

FIG. 1 shows a sagittal profile of known surgery using total anterior lens capsulotomy.

FIG. 2 shows a coronal view of known surgery using total anterior lens capsulotomy.

FIG. 3 shows a sagittal view of endocapsular cataract extraction with segmental capsulotomy according to an embodiment of the invention.

FIG. 4 shows coronal view of endocapsular cataract extraction with segmental capsulotomy.

FIG. 5 shows a number of alternative slit configurations.

FIG. 6 shows the application of a laser to a lens capsule in a first orientation.

FIG. 7 shows the application of a laser to a lens capsule in a second orientation.

FIG. 8 shows a laser docked close to the eye.

### DETAILED DESCRIPTION OF THE DRAWINGS

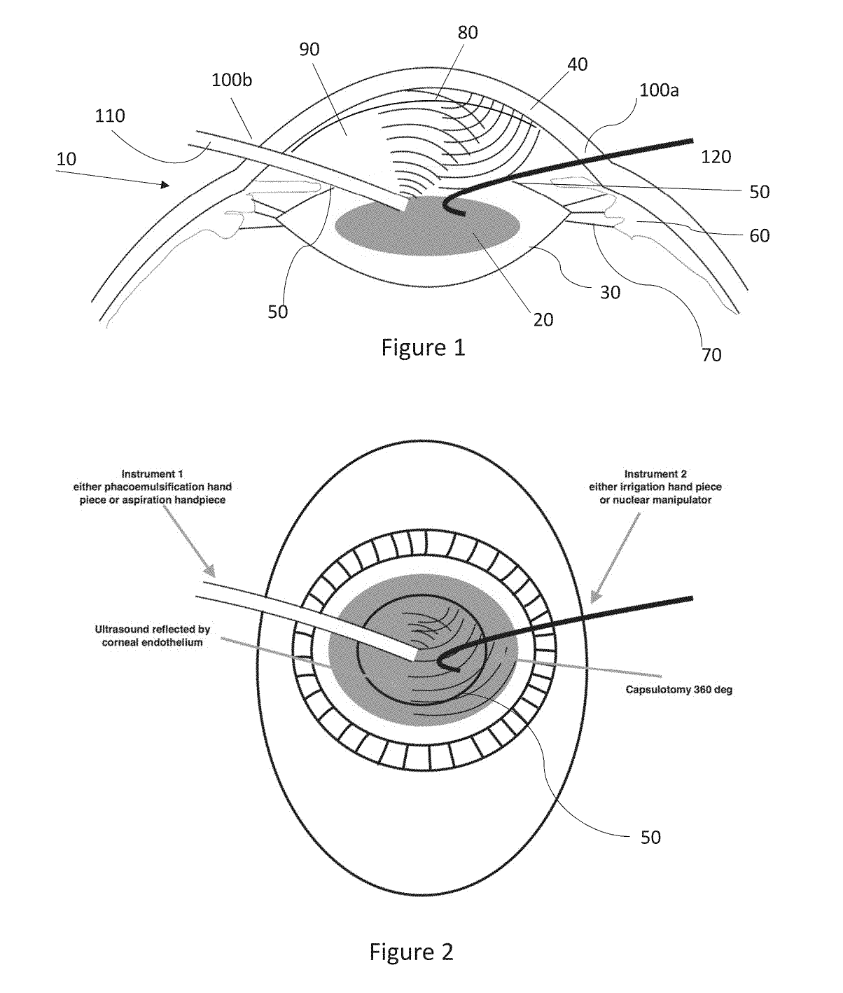

FIGS. 1 and 2 show a known method for removing a lens from an eye using total capsulotomy in the anterior lens capsule. FIG. 1 shows a sagittal profile view through a portion of an eye 10. The lens nucleus 20 is positioned within lens capsule 30.

Lens capsule 30 is connected to ciliary body 60 by lens zonules 70. The ciliary body is a circular muscle within the eye which acts on the lens to control the shape of the lens. The shape of the lens affects the focal length of the lens. When the ciliary body 60 contracts, the tension on lens zolules 70 is released which subsequently releases the tension in the lens capsule. This allows the lens to relax and return to its natural, more spherical, shape. When the ciliary lens relaxes, its diameter is increased and tension is applied to lens zonules 70. This tension pulls the lens capsule. The effect is squeezing of the lens which reduces its thickness and increase its diameter.

The lens is positioned behind cornea 40. On the inside of the cornea is a monolayer of nonreplicating corneal endothelial cells 80. The anterior chamber 90 is positioned between cornea 40 and the lens capsule 30.

Typically, the thickness of the lens is around 3 mm and the depth of the interior chamber around 3 mm. The thickness of the posterior lens capsule (behind the lens) is around 8 microns and the anterior lens capsule is around 20 microns thick.

FIGS. 1 and 2 illustrate a known form of cataract surgery using a total capsulotomy of the anterior lens capsule. In total capsulotomy procedure the anterior central portion of the lens capsule is completely removed. The lens is extracted through the capsulotomy. The lens capsule is cleaned and a replacement lens is inserted in the lens capsule.

As shown in FIG. 1, incisions are made in the cornea at 100a, 100b to provide access to the lens capsule by surgical tools. The anterior central section of the lens capsule is cut away. The circumference 50 of the capsulotomy is illustrated in FIGS. 1 and 2. FIG. 2 shows a front view of the open lens capsule.

Surgical instruments 110, 120 are inserted through the incisions in the cornea and further pushed through the capsulotomy in the lens capsule to access lens 20. Typically, a phacoemulsification hand piece 110 is used to emulsify the lens. Phacoemulsification is performed by applying ultrasound to the crystalline lens. The second surgical instrument, 120 is typically an irrigation hand piece or nuclear manipulator. The second hand piece 120 is used to physically break the lens during the emulsification procedure. After complete emulsification of the lens the emulsified lens is extracted from the lens capsule using suction techniques. The suction is typically applied via a suction tool inserted through the cornea and into the lens capsule via the capsulotomy.

After extraction of the lens a manufactured lens can be inserted into the lens capsule via the capsulotomy to complete the surgery. After surgery, the capsulotomy remains and the lens capsule retains the hole in the front of the capsule.

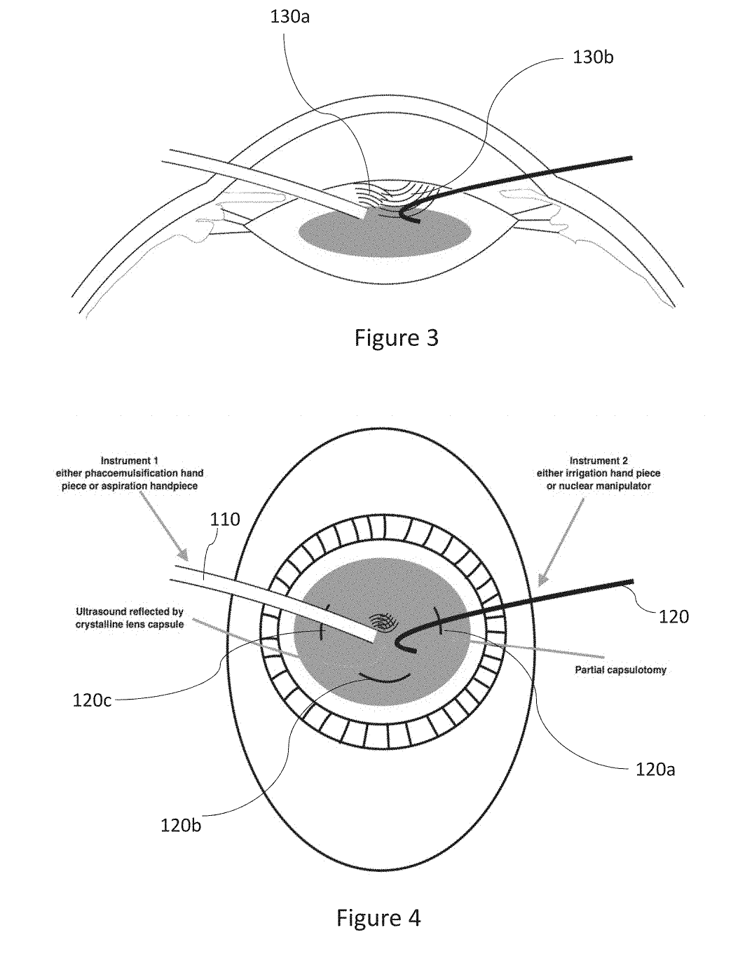

FIGS. 3 and 4 illustrate a method of performing cataract surgery using an embodiment of the invention. The anatomy of the eye in FIGS. 3 and 4 is the same as that in FIGS. 1 and 2 and equivalent components of the eye are identified using the same reference numerals as those of FIGS. 1 and 2.

In the embodiment of FIGS. 3 and 4 access is provided to the lens capsule 30 via the incisions in the cornea 100a, 100b in the same way as the technique described in FIGS. 1 and 2.

In the examples shown in FIGS. 3 and 4, three slits 120a, 120b, 120c are formed in the front of the lens capsule. The slits extend completely through the front surface of the lens capsule to provide access to lens 20. The slits are formed using surgical tools for example a femtosecond laser of appropriate energy, spot and layer separation. In some embodiments the slits are made with a blade. The size and shape of the slits are configured to be suitable enable suitable instruments to enter the slit without stretching or stressing the edges of the slit.

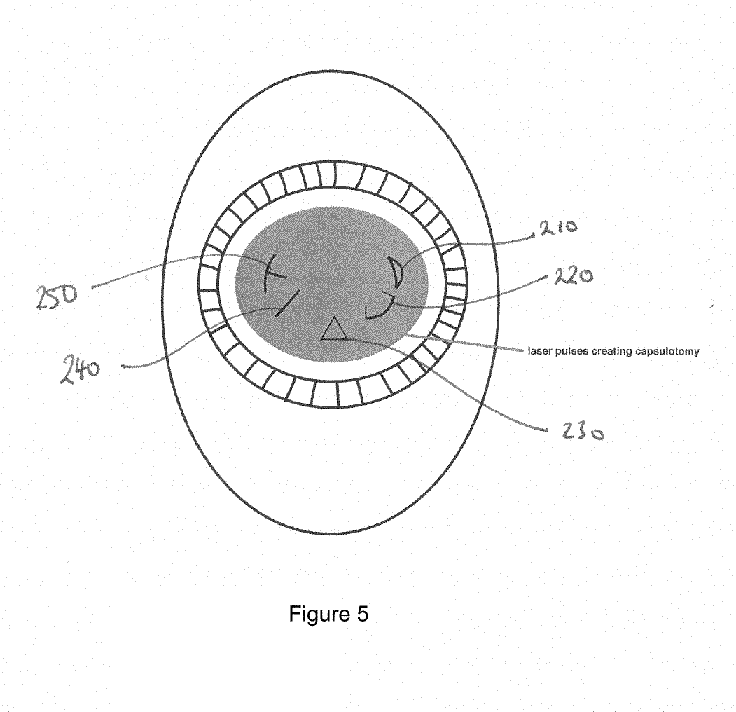

In the embodiments of FIGS. 3 and 4, the slits are arc shaped. In further embodiments the slits are formed in other shapes. FIG. 5 shows examples of alternative shaped slits including mouth shaped 210, arched flap 220, triangular 230, perpendicular to the limbus 240 or T shaped.

The length of the slits in the example of FIGS. 3 and 4 is suitable to receive the necessary surgical instruments. In some embodiments the slits have a length of less than 0.9 mm. One risk during the procedure is the risk of tearing the lens capsule. The incisions are arranged to be larger than the instruments which they receive in order that the edge of the slits are not stretched.

The length slits may be the same length or have different lengths depending on the required surgical instruments to be inserted through the slits.

Slits 120a, 120b, 120c are positioned around and away from the centre of the limbus. In example of FIGS. 3 and 4 the slits are positioned at variable distances from the centre of the limbus. In further embodiments the slits may be positioned at the same distance at a fixed distance from the centre of the limbus.

In some embodiments the slits are positioned radially on the lens capsule this may reduce the impact of reduction in strength of the lens capsule.

In example of FIGS. 3 and 4, three slits are provided in the lens capsule. However, in further embodiments one or more slits may be provided in the lens capsule depending on the requirements of the surgical operation.

In the example of FIG. 4 the slits are positioned at the twelve o'clock, nine o'clock and six o'clock positions. In further embodiments the slits are positioned at other positions around the lens capsule.

In the embodiment of FIG. 4 a phacoemulsification hand piece is inserted through slit 120c and irrigation hand piece 120 is inserted through slit 120a.

At least one of slits 120a, 120b, 120c is sufficiently large to allow evacuation of the nuclear lens material after emulsification.

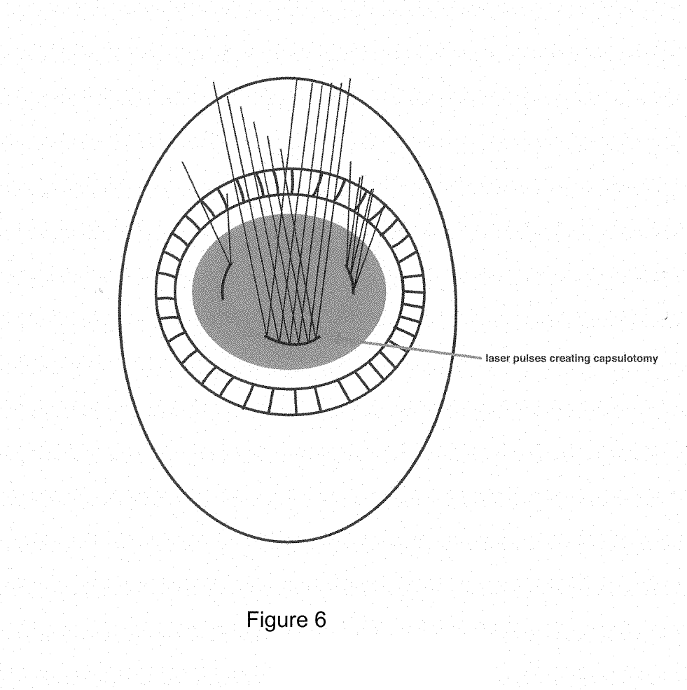

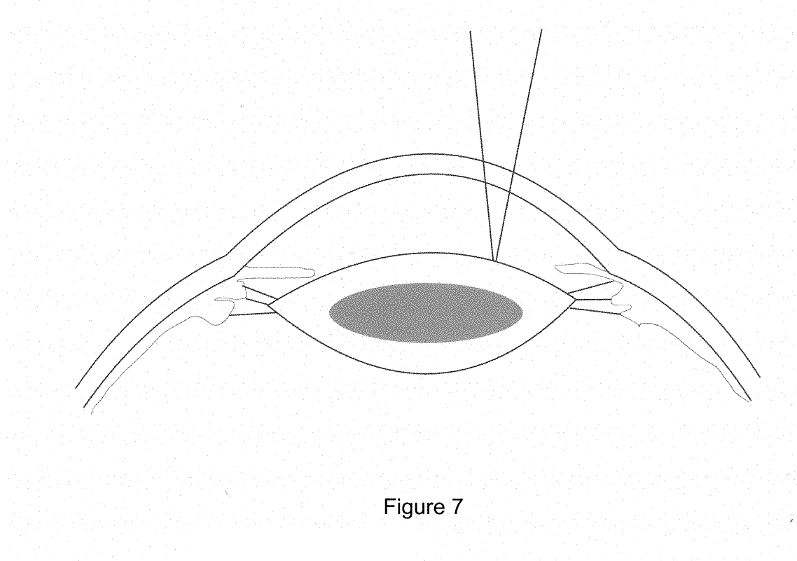

FIGS. 6 and 7 show the application of laser pulses to the lens capsule to create the slits in the lens capsule.

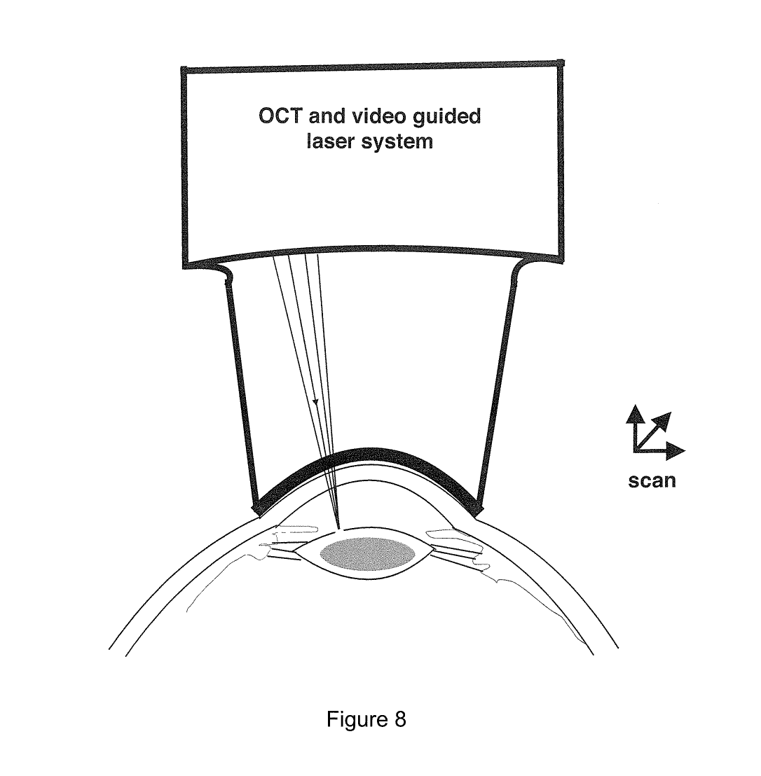

FIG. 8 shows a laser docked close to the eye with laser beams being focused on the anterior capsule. The laser is guided using optical coherence tomography and video guidance to focus the laser beams at different desired locations in the eye.

After removal of the lens an artificial lens can be inserted into the lens capsule through one of the slits to complete the surgery. By utilising the procedure described above, including the slits, a majority of the strength of the lens capsule can be retained. This enables the eye be able to manipulate the artificial lens and remould the lens, i.e. accommodate.

FIG. 3 shows the acoustic wave applied from phaco emulsification hand piece 110. Acoustic wave 130a is dampened by the anterior lens capsule which remains in place throughout the surgery. The acoustic wave is dampened by absorption within the lens capsule and reflection within the capsule 130b. Further dampening of the acoustic wave is provided by the surrounding lens material, the anterior capsule and the soft shell OVD. The dampening limits the exposure of the corneal endothelium to the ultrasound acoustic trauma or trauma from fluidic turbulence. This protection of the corneal endothelium provide a significant advantage compared with known cataract extraction surgeries in which the front portion of the lens capsule is completely removed and the acoustic wave is provided with less dampening before reaching the corneal endothelium.

Further advantages of the technique provide that the anterior chamber will remain filled with OVD material while the instruments are deep within the lens evacuating the hard fragments. This potential offers stable anterior chamber and pain free surgery. In known cataract extraction surgeries the removal of the front portion of the lens capsule can create draining of the OVD material from the interior chamber.

Additionally, the capsule would remain tethered and therefore not float away, making it less difficult to remove if needed. Fluid exchange from the handpieces could create a potential space for the surgeon to operate inside the crystalline lens. This could potentially reduce the use of OVD. As mentioned above the capsulotomy can then potentially be completed carefully to a large circle to potentially allow greater access to the capsular bag if desired.

By maintaining the integrity of the front of the lens capsule, the ability of the lens zonules to apply pressure to the lens after the surgery is maintained. This maintains the accommodation of the lens and helps to retain the control of the focal length by the patient. New lenses which have a liquid semi solid design could be injected and injected in place.

1. A method for removing a lens from an eye comprising the steps of:
making a hole in the outer eye to provide access to a lens capsule within the eye;
cutting at least one discrete slit in the lens capsule to provide access to the lens, the slit being suitable to receive a tool to extract the lens; and
removing the lens through the slit.

2. A method according to claim 1 wherein the at least one slit is positioned away from the centre of the limbus of the eye.

3. A method according to claim 1 wherein the at least one slit is positioned at a fixed distance from the centre of the limbus of the eye.

4. A method according to claim 1 wherein the at least one slit is positioned at a variable distance from the centre of the limbus of the eye.

5. A method according to claim 1 wherein the at least one slit comprises at least two discrete slits.

6. A method according to claim 1 wherein the at least one slit is arched.

7. A method for removing a lens from an eye comprising the steps of:
making a hole in the outer eye to provide access to a lens capsule within the eye;
cutting two or more discrete slits in the eye capsule, the slits being positioned away from the centre of the limbus of the eye, wherein at least one of the slits is suitable to receive a tool for extracting the lens; and
removing the lens through at least one of the slits.

8. A laser configured for use in cataract surgery the laser being configured to cut at least one discrete slit in the lens capsule of an eye to provide access to the lens, the slit being suitable to receive a tool to extract the lens.

9. A laser according to claim 8 wherein the at least one slit is positioned away from the centre of the limbus of the eye.

10. A laser according to claim 8 wherein the at least one slit is positioned at a fixed distance from the centre of the limbus of the eye.

11. A laser according to claim 8 wherein the at least one slit is positioned at a variable distance from the centre of the limbus of the eye.

12. A laser according to claim 8 wherein the at least one slit comprises at least two discrete slits.

13. A laser according to claim 8 wherein the at least one slit is arched.

14. A laser according to claim 8 wherein the laser is configured to emulsify the lens.

15. A method according to claim 1 including the further step of inserting an artificial lens into the lens capsule after removal of the lens.
